(19) United States
(12) Patent Application Publication
(10) Pub. No.: US 2013/0149759 A1
(43) Pub. Date: Jun. 13, 2013

(54) Application of Bacteriophages for the Control of Unwanted Bacteria in Biofuel Production Mediated by Bacterial Reactive Agents

(71) Applicants: Elizabeth J. Summer, College Station, TX (US); Mei Liu, College Station, TX (US)

(72) Inventors: Elizabeth J. Summer, College Station, TX (US); Mei Liu, College Station, TX (US)

(73) Assignee: PHAGE BIOCONTROL RESEARCH, LLC, College Station, TX (US)

(21) Appl. No.: 13/466,272

(22) Filed: May 8, 2012

Related U.S. Application Data
(60) Provisional application No. 61/536,407, filed on Sep. 19, 2011.

(51) Int. Cl. C12P 7/06

(52) U.S. Cl. CPC C12P 7/065
USPC 435/161; 435/235.1

(57) ABSTRACT

A method of reducing process interruptions in biofuel production systems mediated by bacterial reactive agents by reducing the amount of unwanted bacteria without reduction of wanted or useful bacteria. The method comprises applying a phage panel(s) containing phage virulent for unwanted bacteria that does not contain phage phages virulent wanted bacteria. The method includes, optionally, the selective production of solutions of phage for unwanted bacteria from which phages for wanted bacteria are screened out.

### RELATIONSHIP TO OTHER APPLICATIONS

This application claims benefit of and priority from Provisional application 61/539,407 filed Sep. 9, 2011.

### BACKGROUND

1. Field of the Invention

This invention relates to a method of reducing process interruptions in biofuel production systems by reducing the amount of unwanted bacteria in the biofuel production system. More specifically, the reduction is effected by the use of an effective amount of one or more strains of bacteriophages virulent for at least some strains of unwanted bacteria.

2. Background

Biofuel is gaining wide consumer and regulatory acceptance as a renewable fuel. In the U.S., fuel ethanol production has increased from 1.7 billion gallons in 2000 to almost 12.5 billion gallons in 2009 (www.ethanolrfa.org/pages/statistics). The number of ethanol fermentation facilities is also rapidly increasing, from 110 U.S. plants operating in 2007 to 187 in 2010. Biofuel fermentation facilities utilize microbial activities to convert feedstock into ethanol. The majority of commercial biofuel fermentation plants in the U.S. are designed to utilize a grain feedstock, primarily corn, which is fermented by yeast into ethanol. Cellulosic and lignocellulosic feedstocks are attractive alternatives to grain feedstocks, although they present additional challenges in terms of preparing the fermentable substrate.

The fuel ethanol fermentation process is not a sterile process and chronic bacterial contamination at plants is common. While bacterial levels vary during the different steps preparing the grain substrate for fermentation, by the time the processed mash is ready for yeast inoculation, the total bacterial levels in a normal, “healthy” fermentation facility are around 10⁶ colony forming units (CFU) per ml in a wet mill and as high as 10⁸ CFU/ml in a dry-grind facility (Skinner and Leathers 2004). However, bacterial levels higher than this frequently develop, negatively impacting ethanol yields. The most widely cited agents responsible for fuel ethanol fermentation slowdown are lactic acid bacteria (LAB), primarily members of the Gram-positive genera Lactobacillus, Pediococcus, Leuconostoc and Weissella.

While more data is available on the impact of bacteria on grain feedstock utilizing facilities, pilot plants utilizing lignocellulosic feedstock are also subject to contamination by undesirable bacteria (Schell, Dowe et al. 2007). Phages are appropriate for use in controlling unwanted bacterial species in lignocellusosic plants utilizing bacterial fermentation. This is because phages can be used to selectively reduce individual bacterial species from a mixed bacterial environment (Kurtboke and French 2007).

### Current Control Methods

Bacterial control methods have an immediate positive impact and even a simple one-log reduction in the amount of LAB can increase ethanol yield by approximately 3.7% (K. Bischoff, pers comm., Bischoff, Liu et al. 2009). Bacterial contamination in fuel ethanol plants is typically controlled by a combination of plant management approach and through the addition of chemical antimicrobials and antibiotics.

Challenges Associated with Antibiotic Use

Not surprisingly, antibiotics, in particular virginiamycin and penicillin, have been found particularly effective in curbing bacterial populations without disturbing the system reactive agent culture in yeast-based fermentation systems. However antibiotic are not selective and cannot be used in lignocellusosic plants utilizing bacterial fermentation. In these plants the LAB bacterial must be selectively destroyed (or reduced) without disrupting the beneficial and fermentation bacteria.

Biocides are virtually removed as an option, and antibiotics must be carefully selected so as not to kill the system bacteria. Inhibitory compounds designed to control unwanted bacteria are also much more likely to affect system bacteria than another agent, such as yeast. Controlling the environment likewise becomes much more challenging when the problem and the system agent are so similar. Therefore, a method of selectively applying phages virulent for one or more unwanted bacteria, yet not virulent for the system reactive agent(s), is needed. The present invention is designed to meet that need.

The present invention is an entirely new approach to control unwanted bacteria in biofuel production processes in which bacterial system reactive agents are utilized, of which control of unwanted LAB in the fuel ethanol fermentation industry, based on LAB bacteriolytic phage formulations, is a preferred aspect.

### Historical and Current Commercial Phage Use

Phage themselves are not new, having been discovered during the First World War. The most obvious use of phages is for medical applications. While early interest in phage therapy was suppressed by the introduction of antibiotics, the recent rise in antibiotic resistance and costly food contamination events has led to a resurgence of interest in phages (Kropinski 2006; Mattey and Spencer 2008; Housby and Mann 2009). In the United States, phages have been applied on human patients as part of a more comprehensive approach to controlling and curing chronic wounds associated with diabetic ulcers and pressure wounds. In 2007, phages were approved by the FDA as a food additive, specifically for the control of the food-borne pathogen Listeria on commercial luncheon meats (Bren 2007). Commercial phage products sold in the U.S. include AgriPhage, sold by Omnilytics and designed to control Xanthomonas infestations in peppers and tomatoes and Finalyse, sold by Elanco Foods and designed to control E. coli O157:H7 levels on slaughterhouse cattle.

### Need for Phage Treatment of Bacterial System Organisms

This invention will have immense and unparalleled potential for reducing ecological and environmental impact of human activities through the removal of specific microbial contaminations in biofuel production facilities without the use of antibiotics, and provides a green alternative to toxic chemical biocides and antibiotics for controlling problem bacteria in the industrial biofuel production industries.

### SUMMARY OF THE INVENTION

The invention described herein is a method of reducing the concentration of unwanted bacteria in biofuel production processes which utilize bacterial system reactive agents. One or more bacteriophage panels, which are comprised of one or more bacteriophages virulent for one or more strains of the unwanted bacteria, are applied to some process point of the biofuel production process to reduce the concentration of unwanted bacteria.

Phages are natural bacteriolytic agents suitably used to selectively reduce individual bacterial species from a mixed bacterial environment. This invention will result in improved fermentation efficiencies, reduced wastes, and reduced antibiotic residue in solid byproducts. The improved bacteria control methodology of this invention is easily incorporated into current biofuel production systems—particularly ethanol fermentation plants—is non-toxic, has no negative effect on the bacterial cultures (such as fermentative Clostridial, Z. mobilis, or E. coli cultures, or other genetically modified bacteria, including LAB strains), and has competitive economics.

In broad aspect, the invention is a process for control of unwanted bacteria in a biofuel production process comprising a reaction mediated by bacterial reactive agents. In a more preferred embodiment, the biofuel production process is an ethanol production process comprised of fermentation of suitable feedstocks, wherein the control of unwanted bacteria is effected by bacteriophages assembled into one or more bacteriophage panels such that one or more bacteriophage strains in each panel is virulent for one or more unwanted bacteria. Suitable bacteriophage may be resident in the biomass feed to the process or located and identified from other sources.

In another embodiment, the invention is a dynamic phage multi-panel produced by repetitive or continuous proliferation, concentration, or both proliferation and concentration of bacteriophages resident in the biomass, which may be conducted onsite or at a central location.

In another embodiment, treatment to control unwanted bacteria in the biofuel production process may be conducted by bacteriophages in various ways, before operation, during operation or as a prophylactic for cleaning process vessels and equipment.

The invention is also a composition comprising an assembly of one or more bacteriophages virulent for unwanted bacteria in a biofuel production process.

The invention is also a process for control of unwanted bacteria in a biofuel production process which utilizes non-bacterial system organisms, comprising concentrating one or more strains of bacteriophages resident in the biomass to an effective concentration used for control of one or more strains of unwanted bacteria.

### DESCRIPTION OF THE DRAWINGS

FIG. 1 is a diagrammatic representation of embodiments of the process of the invention.

FIG. 2 is a diagrammatic representation of embodiments of the process of the invention.

FIG. 3 is a diagrammatic representation of embodiments of the process of the invention.

### DETAILED DESCRIPTION OF THE INVENTION

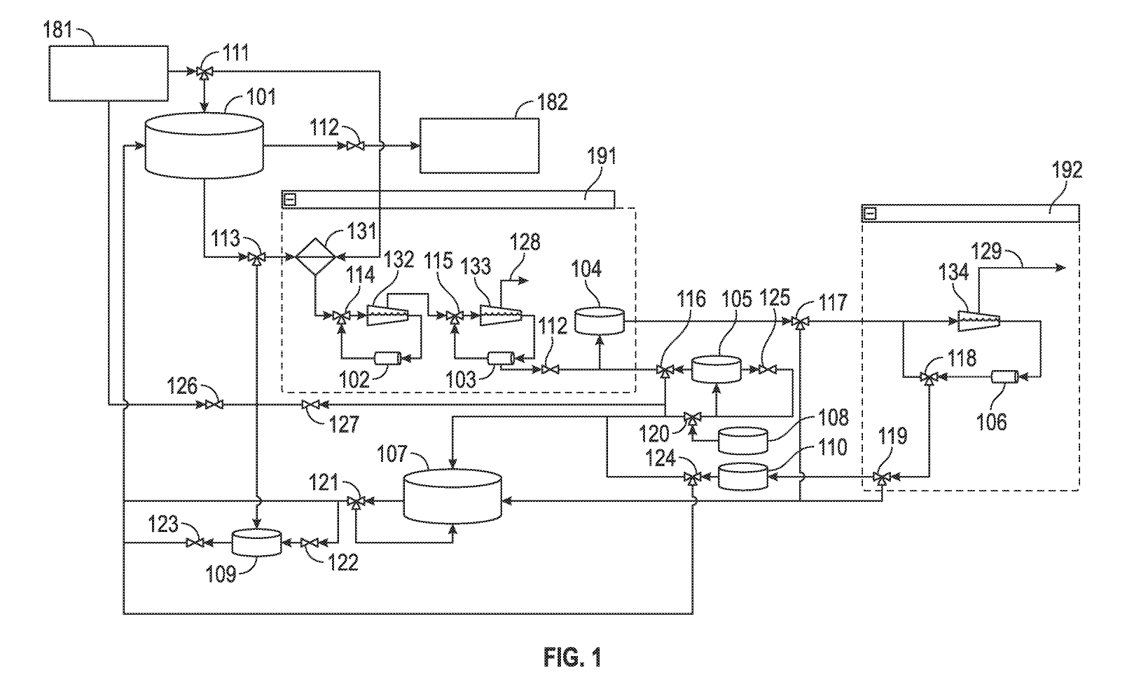

The present invention is a method of utilizing bacteriophages to selectively control unwanted bacteria in bacteria-mediated reaction processes involved in biofuel production. In a preferred aspect, the method is applied to control unwanted lactic acid bacteria (LAB) in the bacteria-based fermentation stage of ethanol production. As illustrated in the upper left hand corner of FIG. 1, the basic ethanol plant process is depicted as a biomass stream from the previous production step 181 (e.g. hydrolysis) flowing into reactor 101 (e.g. a fermentation tank) through valve 111 and out again to the next production step 182 through valve 112. The invention is comprised of various process embodiments and systems for producing and applying phages to the production process units, particularly fermentation. The invention selectively targets unwanted bacterial strains which may be interfering with the production process, while not, to any significant degree, negatively impacting system bacteria used in the biofuel production process.

### Preferred Aspect of Fuel Ethanol Fermentation

In one preferred embodiment of this invention, panel(s) of virulent phages specific for unwanted bacteria are utilized to reduce the load of fermentation failure-associated LAB.

In a preferred embodiment of this invention, phages are used to control unwanted bacterial species in sugar/starch and lignocellusosic feedstock ethanol plants utilizing bacterial fermentation. In a more preferred embodiment, the unwanted bacterial species targeted are from acetic and lactic acid producing genera, especially those referred to as lactic acid bacteria.

Although the present invention is focused on treating LAB in fermentation in particular, and bacterially-driven fermentation processes in general, it will be clear to those skilled in the arts of microbiology, biofuel production, and related fields, that the invention may be applied to any similar biofuel production process, so long as: 1) the process is driven by one or more phage-sensitive system reactive agents, and 2) it is desirable to control one or more unwanted bacterial strains. Examples of alternative embodiments include, but are not limited to, controlling unwanted bacteria in biofuel production using cyanobacteria (known also as “prokaryotic algae” or “blue-green algae”) for their production of sugars and cellulose and production of cellulases by bacteria (specific examples of bacterial strains used include Acidothermus cellulolyticus, Bacillus sp, B. subtilus, Clostridium acetobutylicum, C. thremocellum, Pseudomonas cellulosa, and Rhodothermus marinus) for immediate or later use in biofuel production.

### DEFINITIONS

As used herein, it is understood that the terms “phage(s)” and “bacteriophage(s)” are synonymous. Other terms used herein have the meaning stated below.

### Biomass

The term “biomass,” as used herein, refers to “biological material derived from living or recently living organisms The term is often used to mean plant based material, but biomass can equally apply to both animal and vegetable derived material.” Biomass may include, but is not limited to, grain feedstocks (e.g. corn), high-energy feedstocks (e.g. sugar cane), cellulosic and lignocellulosic feedstocks, and plant or animal waste feedstock (including bagasse and other wastes from cellulosic, sugar, and starch biomass used for fuel production or some other process). In the context of this application, the term “biomass” is also understood to refer to any mass, resulting from the original biomass, downstream of the original process. One non-exclusive example would be a lignocellulosic feedstock being referred to as “biomass,” while the term “biomass” is also used to refer to the mixture during and after hydrolysis, fermentation, etc. The term “biomass stream,” as used herein, refers to biomass as it moves through a biofuel production system, from the origination of a particular system, through and between each process, and concluding with the final product(s) and waste products.

### Bioreaction

The term “bioreaction,” as used herein, refers to the use of one or more living organisms or enzymes (“system reactive agent(s)”), to convert one or more substrates into one or more products. Examples include, but are not limited to: yeast or bacteria (“system reactive agent”) metabolizing sugars (substrates) and producing ethanol (product), bacteria (“system reactive agent”) metabolizing a feedstock (substrate) and producing enzymes (product) used in some aspect of biofuel production, or an enzyme (system reactive agent) used to convert carbohydrates (substrate) into sugars (product).

As used herein, the term “reaction” is understood to include, but is not limited to, both chemical reactions and bioreactions.

### Bioreactor

The term “bioreactor,” as used herein, refers to a vessel, tank, vat, or other container in which one or more bioreactions are performed. As used herein, the term “reactor” is understood to include, but is not limited to, tanks, vats, vessels or containers used for performing chemical reactions and/or bioreactions.

### Biofuel

The term “biofuel,” as used herein, refers to any fuel produced from biomass feedstocks. Examples include, but are not limited to: bioethanol; biobutanol; hydrocarbon bio-gasoline, diesel, or jet fuel (drop-in fuels); biodiesel; chars and tars; bioliq; biomethanol, and biohydrogen. The term is used regardless of production process.

### Biofuel Production System and Process

The term “biofuel production system,” as used herein, refers to a system, or any part thereof, directly or indirectly involved in the production of biofuel. Examples include, but are not limited to, an ethanol production plant, a cyanobacteria-based biofuel plant, and an enzyme production process where at least one use of the enzymes produced is utilization in some aspect of biofuel production. Accordingly, it is understood herein that the term “biofuel production process” is used to refer to any process or processes used by a biofuel production system.

### System Reactive Agent

The terms: “reactive agent,” “system reactive agent,” “system organism,” “system microbe,” “system microorganism,” or “system bacteria,” as used herein, refer to the agent(s) responsible for the bioreaction in the particular biofuel production system or process. The system reactive agent(s) need not necessarily be known, isolated, or identified; the sole defining characteristic is that it is the reactive agent(s) desired, ideal, and/or necessary for the desired bioreaction(s) to proceed. In this invention, system reactive agents are bacteria.

### Unwanted Bacteria

The term “unwanted bacteria,” as used herein, refers to the strain(s) of bacteria specifically targeted for control by the invention described herein. Typically, but not necessarily, the unwanted bacteria is targeted for control because of interference with the reaction(s), such as in the case of unwanted LAB in ethanol fermentation. The unwanted bacteria need not necessarily be known, isolated, or identified; the sole defining characteristic is that it is the organism(s) desired to be controlled. This invention provides for reduction of invasive bacteria and other unwanted and problematic bacteria.

### Phage Panel(s) and Cocktail(s)

The term “phage cocktail,” as used herein, includes multiple, receptor independent phages for a single targeted strain of unwanted bacteria. This is different from a “phage panel,” which is a collection of phages chosen to target a particular range of hosts. For the purposes of this invention, the phage treatment may be comprised of one or more “phage panels,” each comprised of one or more “phage cocktails,” that is, there will be one or more virulent phage strains targeting each unwanted bacterial strain and one or more phage cocktails targeting one or more unwanted bacterial strains. Since some phages are known to be polyvalent—effective against more than one strain of bacteria—there may not always need to be a separate cocktail for every strain of unwanted bacteria.

Therefore, as used herein, it is understood that “phage panel(s)” refers in the broadest sense to a combination of one or more phage(s) intended for control of one or more bacterial strains. It encompasses everything from one phage cocktail comprised of one phage strain, to many phage cocktails, each comprised of many phage strains. A “phage multi-panel” refers to one or more phage panels, or the total suite of phage strains used in a particular application.

### Assembly

The terms “assemble, “assembly,” “assembled,” and their related forms, as used herein in connection to phage cocktail(s), panel(s) and multi-panel(s), refer to choosing and compiling the strain(s) of phages making up the panel(s). This need not include actual identification or isolation of the phages, however, such as in the case of dynamic phage multi-panels discussed hereafter. Assembly of a phage panel does not refer to production or proliferation of the panel.

### Proliferation

The terms “proliferate,” “proliferating,” “proliferation,” and their related forms, as used herein in connection to phages and phage cocktail(s), panel(s), and multi-panel(s), refer to the reproduction (growth or increase in quantity) of the phage(s) being referenced by infection and lyse of host bacteria. As used herein, the terms are not meant to describe speed of growth or reproduction.

### General Description of Method

The fundamental innovation outlined in this invention is use of bacteriophage based formulations for the control of unwanted bacteria in the biofuel industry. Phages are remarkably abundant in the environment, even more so than bacteria. Phages are naturally abundant in many food products and are therefore routinely consumed. Because of their ubiquity, specificity for bacterial cells, and their lack of interaction with human, animal, or plant cells, phages have been designated by the FDA as generally regarded as safe (GRAS).

In simplest terms, application of any phage based antimicrobials can be divided into four distinct processes.
1. Identification of unwanted bacteria in the target process.
2. Assembly of one or more phage panels or multi-panels, each of which is comprised of one or more virulent phage types active against the unwanted bacterial strain(s) targeted and inactive against wanted or beneficial bacterial strains.
3. Large scale phage production and processing into application form, and
4. Application of the phage panel(s) to selectively control the unwanted bacteria in the target process.

### 1. IDENTIFICATION OF INVASIVE BACTERIA

The first step of the invention is to identify problem (unwanted) bacteria, in order to be able to isolate and propagate virulent phages against them.

### Selection of Target Strains, Exemplified by LAB in Fuel Ethanol Fermentation Plants

Many LAB phages have been identified and isolated, but most are those virulent against dairy associated LAB. While closely related, dairy and fuel ethanol fermentation LAB strains are not identical. Due to the specificity of phages, it is usually preferable that bacterial strains be used that have been isolated from fuel ethanol fermentation plants, especially those that have been demonstrated to reduce fermentation efficiencies. For example, in ethanol fermentation affected by LAB, Drs. Bischoff, Leathers, and Rich have identified 200 isolates of Lactobacillus species collected from commercial ethanol facilities (Skinner and Leathers 2004; Bischoff, Skinner-Nemec et al. 2007). Five example phage target strains are: L. fermentum 0315-1, L. fermentum 0315-25, L. brevis 84, L. mucosae 0315-2B, and L. amylovorus 0315-7B. This collection represents the more common yet genetically distinct Lactobacillus species isolated as contaminants from the fermenters of commercial ethanol facilities experiencing acute contamination problems. Furthermore, all but L. amylovorus 0315-7B have been shown to produce stuck fermentations in shake-flask models of ethanol fermentation (Bischoff, Skinner-Nemec et al. 2007). L. fermentum 0315-1, L. fermentum 0315-25, and L. brevis 84 were isolated from planktonic cultures, while L. mucosae 0315-2B and L. amylovorus 0315-7B were associated with biofilm cultures.

In addition to Lactobacillus species, other bacteria of interest include, but are not limited to, other lactic acid producing bacteria, such as species in the Pediococcus, Lactococcus, Enterococcus, Weissella, Leuconostoc, Streptococcus, and Oenococcus genera, and acetic acid producing species, such as the Acetobactor and Gluconobacter genera. Additional species of unwanted bacteria, including those that are discovered to particularly affect various bacterial fermentation processes and those affecting processes other than fuel ethanol fermentation, are also target species and will be evident to those skilled in the art.

### Sources of Bacteria Populations

Unwanted bacteria are identified by sampling the source biomass and/or biofilm. From samples, the unwanted bacteria can be isolated and characterized, to some extent based on what is generally already known about the causes of the undesirable effects, e.g. stuck fermentation. From these samples, virulent bacteriophages are identified for target unwanted bacteria, e.g. LAB. Sufficient phages are then isolated to effectively lyse the unwanted bacteria, and an effective amount of phage solution is added to the biomass. Isolation and identification of phages is discussed in following sections.

Multiple bacterial populations may work synergistically. Members of microbial consortia exhibiting biofilm formation activity, for example, may provide the anaerobic microenvironment preferred for the growth of LAB. As such, the target of phage treatment, and therefore, the target unwanted bacteria, can include not just the bacteria competing with and/or inhibiting the system reactive agents, but also any bacteria involved in forming the microenvironment required or contributing to their proliferation.

Therefore, biofilm producing bacteria involved in inhibiting the system process are included in the category of targets for phage remediation. Biofilm forming genera of bacteria include Pseudomonas or Vibrio species isolated in affected systems.

Bacterial populations responsible for biofilm resulting in physical blockage in the production process may also be selected for phage treatment. All bacteria that are to be targeted for phage treatment are part of the selected bacterial subpopulation.

### Laboratory Culturing of Unwanted Bacterial Strains

Lactobacillus species may be grown as static cultures in MRS media (Difco Lactobacilli MRS Broth) or as colonies on solidified MRS agar plates at 37° C. Although anaerobes, Lactobacillus species are aerotolerant so cultures may be set up on the bench before transferring to anaerobic chambers for growth. Lactobacilli may be grown in simple GasPak jars or in functional anaerobic chambers, which permit easy manipulations and assessment of anaerobic microorganisms.

### 2. ASSEMBLY OF PHAGE PANEL(S)

### Sources of Phages

In a preferred embodiment, phage isolation is achieved through an enrichment procedure.

Enrichment is an effective means for isolating new phages, as if even a single phage is present in the starting solution it is not unreasonable to obtain a final phage concentration of 10⁷ or 10⁸ plaque-forming units (PFU; corresponds to active phage particles) per ml solution (Brownell, Adams et al. 1967; Brownell and Clark 1974). As a predator, natural populations of phages are found near natural populations of their prey. Therefore, an important consideration in choosing samples is to choose them from sites where the host bacteria can be found.

The sources of phages for controlling bacterial infestations include any site where bacteria are found. While existing phage stocks may be screened for activity on unwanted bacteria, new phages will normally also be isolated from the same site or location where the bacteria pose a problem, such as fermentation mash (or biomass in general), deposits in system containers, and the like.

Populations of bacterial phages will be most abundant near abundant sources of their prey. Therefore, the process of identifying phages specific for any bacterial population is to first identify an environmental site where that bacterial type is abundant. This means that there is not one environment that will serve as a source of phages for all target microbes. Instead, the exact environmental sample will vary from host strain to host strain. However, there are general guidelines for identifying the environmental sample most likely to yield desired phages. An ideal starting-point sample will often be biomass from the affected biofuel production plant. However, it is possible that the phage(s) most efficient at destroying a specific unwanted bacteria may not be those naturally co-resident with said strain(s) in the production plant. Therefore, ideal samples may also come from a production plant employing a similar process under similar conditions, or a completely different location, selected following the guidelines discussed herein. Specific physiochemical properties of the biomass are important and exact parameters will vary from host to host. An example, which is not intended to be a guideline for all protocols, would be the identification of phages active against an LAB strain. Biomass enriched in LAB is typically characterized by elevated levels of lactic and/or acetic acid. Therefore, a sample likely to possess LAB specific phages will be biomass that is fermentable, fermented, or a product of fermentation. Especially likely would be a fermenting material rich in sugars (a nutrient source for both the LAB and the system reactive agent(s), such as Clostridium ljungdahlii, Escherichia coli, or Zymomonas mobilis) and with elevated levels of lactic and/or acetic acid (metabolites of LAB). Phage isolation sources may include various liquids and mash solids, obtained from biofuel production facilities of interest. In one embodiment of the invention specifically targeting LAB, fuel ethanol fermentation facilities or any fermenting feedstock may be potential sample sources. Examples include, but are not limited to, fermented dairy products, such as yogurt and cheeses such as feta, and sour foods, such as sauerkraut, pickles, kimchi, and salami. In general, fermenting foods with a “sour” taste indicate LAB activity, and a potential source of phages. Yogurt, for example, has been demonstrated to contain phages active against Lactobacillus isolates from non-dairy environments (Tao, Pavlova et al. 1997).

Phages for any given host can be found at the same conditions that are favorable to the growth of the host bacteria. Bacteria vary greatly with regard to carbon source utilization, similarly phages that infect these bacteria can be found in these environments regardless of carbon source being utilized by the bacteria. Similarly, bacteria and phages vary greatly with regard to tolerance and utilization of industrial waste materials such as metals, heavy metals, radioactivity, and toxic chemical wastes including pesticides, antibiotics, and chlorinated hydrocarbons.

As an alternative to identifying samples based on physiochemical properties, molecular tools are used to identify sediments possessing wild populations of bacteria similar to the unwanted bacteria. These methods typically require some level of purification of DNA from the environmental sample, followed by the detection of marker DNA sequences.

The most straightforward of these are polymerase chain reaction (PCR) based technologies that target 16s rDNA sequences. These can be analyzed by methods such as denaturing gradient gel electrophoreses (DGGE) or by DNA sequencing.

### Laboratory Isolation of Phages

In one embodiment, the first step to identifying phages in a sample is to prepare a sterile-filtered rinsate. To do this solids and bacteria are separated by a combination of centrifugation and filtration. The rinsate, which usually has only a few individual phages against any specific host, is then supplemented with MRS media and inoculated with low levels of the specific target host. The sample is incubated for a period of overnight to several days, depending on the host growth characteristics. At this point, the liquid culture may or may not show evidence of phage activity. Chloroform is added to 0.1% v/v in order to complete lysis of infected but un-lysed cells and phages are separated from bacterial cells and debris by centrifugation and filtration. This is the phage enrichment and may contain more than one type of phage against the target host. The presence of phages is determined by spot titering onto agar overlays containing confluent lawns of the host. It should be noted that many Lactobacillus species produce bacteriocins, which also produce clearings when undiluted supernatants are spotted onto lawns in agar overlays (Tao, Pavlova et al. 1997). Bacteriocins, however, are easily distinguished from phage in that they do not produce plaques following serial dilutions.

If clearings are observed, serial dilutions of the enrichment samples are plated in overlays to the point that individual, well-separated plaques are formed. To generate clonally pure phage stocks, agar plugs containing the well-separated plaques are excised from the plate and phages are eluted from the agar plugs. This process, termed plaque purification, is repeated at least one more time. At this point, the small volume (1 ml) of phages from a single plaque is considered to be clonal and the process of phage amplification can begin. Two approaches, the liquid lysate or plate lysate, approaches may be used to generate high volume, high titer lysates. In the plate lysate approach, overlays are prepared with between 10⁴ to 10⁵ PFU of phages, which produce nearly confluent lysis of the overlay. Phages are eluted from the top agar and purified by centrifugation and filtration. To prepare liquid lysates, the optimal multiplicity of infection (MOI) for maximum phage production must be first determined in a series of preliminary experiments. Then, large volume liquid lysates are prepared by inoculation with the optimal host/phage levels. After growth and lysis of the culture is observed, phages are purified by a combination of centrifugation and filtration.

Phages may be characterized minimally by host range analysis on the collection of species (for example, Lactobacillus) isolated as described above, as well as by restriction digest analysis of genomic DNA. This will provide enough information to cluster the phages into similarity groups, which may reflect host-receptor specificity. More extensive characterization may be completed if necessary or desired.

### Laboratory Characterization of Efficacy of Isolated Phages

In this embodiment, once a collection of phages active against the fuel ethanol fermentation-inhibiting Lactobacillus strains have been assembled, the next step would be to determine efficacy of phage clearing of the hosts in batch cultures. Batch cultures are performed by inoculating liquid media with low levels of the bacterial strains of interest and incubating for a period of several days. Batch culture growth follows classic, single-step growth kinetics and can be broken down into three phases, lag, log, and stationary phase. Lag phase corresponds to the early acclimation of the inoculum to fresh growth media, log phase corresponds to the most rapid period of bacterial division, and stationary phase corresponds to the phase when limiting nutrients are depleted and cell division rates decline or cease. Bacterial growth is monitored by changes in optical density (OD₆₀₀nm). One OD₆₀₀ₙₘ of Lactobacillus sp. is equivalent to approximately 1×10⁸ CFU/ml, where CFU=colony forming units. In the most preliminary types of phage efficacy experiments, phages are added to the bacterial culture at several different MOI (multiplicity of infection, that is the ratio of phage to host cells), typically a MOI of 0.01, 0.1, 1.0 and 10.0. If enough phages are produced, a MOI of 100 will also be tested. Culture ages corresponding to lag, log, and stationary phase cells will be challenged with phages at each MOI. Control cultures, unchallenged by phages, will be analyzed in parallel. The phage effect is typically monitored by measuring changes in host cell OD₆₀₀ₙₘ, enumerating produced phages using the overlay method, and enumerating viable host cells in the culture using the colony counting method. Care must be taken to remove free phages prior to host cell plate counting. Experiments would ideally be repeated in triplicate and quantification of individual time points performed in duplicate. Statistical comparisons of challenged and control cultures may be performed using Student's t-test (P<0.05).

Once batch culture efficacy test results are completed, the next step in this embodiment is to determine phage efficacy in a shake-flask fermentation model system containing both the system reactive agent and the inhibitory unwanted bacteria strains. These experiments are conducted by co-culturing the system bacteria and Lactobacillus in a corn mash. Phages are applied at different MOI and at different times. Appropriate controls run in parallel include co-cultures not challenged with phages, system bacteria only cultures, and unwanted bacteria only cultures. At different time points, bacterial and phage densities will be enumerated by colony and plaque counting, respectively, as described. Levels of ethanol, glucose, lactic acid, and acetic acid may be determined, for example, by high performance liquid chromatography (HPLC) using a 300 mm Aminex HPX 87H column (Bio-Rad Laboratories, Inc., Hercules, Calif.). For this, 10 ml samples are injected onto a heated column (65° C.) and eluted at 0.6 ml/min using 5 mM H₂SO₄ as mobile phase. Concentrations are reported as mean values (±standard deviation) of at least triplicate cultures. Statistical comparisons of challenged and control cultures may be performed using Student's t-test (P<0.05).

### Assembly of Identified Phages

Once suitable phage strain(s) have been identified, phage cocktail(s), panel(s), and multi-panels may be assembled. In one embodiment, standard phage panel(s) may be assembled which are designed for use with one or more application profiles, including, but not limited to: certain feedstocks, environments, production processes, fuel products, system reactive agents, or geographical locales. These pre-assembled phage panel(s) may be stored as a sort of “phage library” that can be stored onsite or offsite (e.g. at a central laboratory/manufacturer) and accessed for rapid response to infestations. In another embodiment, standard phage panel(s) may be custom assembled for a particular situation. In a preferred embodiment, these approaches may be used in conjunction. For example, a standard phage panel(s) may be utilized for rapid response, and then replaced/combined with a more specific custom phage panel(s) once it is prepared.

### 3. PRODUCTION OF PHAGE PANEL(S)

### Proliferation of Isolated Phages

Once suitable phages are isolated and phage cocktail(s), panel(s), and/or multi-panels assembled, they generally will be proliferated to produce a suitable quantity for biomass treatment, and processed as required for the desired application or destination. This may be accomplished onsite at the biofuel facility, or offsite at an external lab or production facility with the resulting quantities of phage panel(s) packaged, stored, and/or shipped as needed. Since many strains of phages are notoriously hardy, they may be concentrated, freeze dried, desiccated, and stored for long periods of time without loss of effectiveness. Phages may be suspended in a medium suitable for application, such as a substance that adheres to vessel walls for use in treatment of process vessels. The solution containing the phages may be filtered to concentrate and/or isolate the assembly of phages. Phages may also be encapsulated with a water-soluble coating. This allows phage cocktails, panels, and multi-panels to be shipped to remote locations for use, and allows the manufacture to be made at optimized central locations. While, in the preferred embodiment, the phage panel(s) are produced “on location,” it is sometimes preferred that the manufacture of large volumes of phage panel be centralized in locations where the necessary equipment and resources are readily available. Alternatively, phage panel(s) may be processed for storage before proliferation.

A preferred embodiment of the invention is also illustrated in FIG. 1. Valve 116 releases unwanted bacteria from storage tank 105 into phage concentration tank 107. Valve 120 releases growth media from storage tank 108 into tank 107 to feed the unwanted bacteria and allow them to replicate and proliferate. Valve 124 releases phage panel(s) from storage tank 110 into tank 107. The phages then infect the unwanted bacteria in the concentration tank and reproduce. Three-way valve 121 delivers the resulting mixture to the reactor 101 or through valve 122 into treatment tank 109. A portion of the mixture may be recycled back into the phage concentration tank to provide a continuous supply of virulent phages. Residence time and recycle ratio may be adjusted to control the concentration of phages in the phage concentration tank. This assembly and process may be referred to as a “phage proliferator/concentrator,” and may be used either onsite or offsite.

### Isolation and Proliferation of Resident Phages

Alternatively, phages can be produced without identification by simply proliferating resident phages directly from the target biofuel production process. One embodiment of this aspect of the invention is illustrated in FIG. 1. Biomass stream is diverted from reactor 101 through three-way valve 113 and passed to subsystem 191 (delineated by dotted lines and marked 191) for filtration. Biomass stream entering subsystem 191 passes through filter 131 to remove debris and “trash.” The stream then passes through three-way valve 114 and through tangential flow filter (TFF) 132, a micro-filtration TFF intended to separate bacteria and phages. The retentate contains bacteria and unfiltered phages, and is recycled through container 102 and valve 114 and back through TFF 132. The filtrate from TFF 132 contains phages present in reactor 101 and is passed through three-way valve 115 and through TFF 133, an ultra-filtration TFF intended to concentrate viruses. This filtration step is designed to concentrate the phages present in the filtrate of TFF 132. Removal of the bacteria is necessary to the success of the following steps. If non-target bacteria were allowed to remain with the indigenous phages, then during the proliferation phase phages virulent to the system bacteria (i.e. those bacteria essential for the success of the process in the bioreactor) would also proliferate and destroy the system bacteria. The filtrate of TFF 133 will be sterile water, removed through conduit 128, while the retentate will be a phage solution. The retentate of TFF 133 is recycled through container 103 and valve 115 and back through TFF 133 until the phages are as concentrated as desired. Typically, a sufficient concentration is above 10⁴ PFU, with a concentration above 10⁵ PFU being preferred, and a concentration above 10⁷ PFU being especially preferred; however, the target concentration will vary widely based on many parameters, including time constraints, phage strains being concentrated, etc. The resulting retentate (after sufficient recycling) from TFF 133 contains a concentrated solution of indigenous phages present in reactor 101.

In an alternative embodiment, TFF 133 can be eliminated and the filtrate of TFF 132 used directly. The resulting phage solution will be less concentrated than if TFF 133 is used. This embodiment may be desirable in cases when reduction in time, labor, and/or expense is required.

The phage solution (retentate from TFF 133 or filtrate from TFF 132) is then passed into incubation tank 104. Target bacteria from storage tank 105 is released through valve 116 and then mixed with the phage solution in incubation tank 104. The resulting mixture is allowed to incubate a length of time sufficient for phage adsorption to the bacteria. Sufficient times will vary depending on the strains of phages and bacteria involved, as well as on other factors, and choosing such suitable values is well within the ability of those of ordinary skill in the art of microbiology.

The incubated mixture is then passed through valve 117 into proliferation/concentration tank 107, where target bacteria is added from tank 105 and growth media is added from tank 108 to facilitate bacteria replication in order to supply hosts for phage proliferation. As the unwanted bacteria concentration rises, and the bacteria infected while residing in incubation tank 104 burst and release phages virulent for the unwanted bacteria, the phages will infect the unwanted bacteria and reproduce, increasing the quantity of phages virulent for the unwanted bacteria. Alternatively, unwanted bacteria may be proliferated and/or concentrated directly in tank 105 by the addition of growth media from tank 108 through valve 120. The target bacteria may then be distributed as discussed elsewhere in this description. A portion of the outflow of tank 105 may be recycled through valve 125 in order to increase the concentration of the target bacteria in the mixture in tank 105. The volume recycled and the residence time in tank 105 may be adjusted to control concentration and to provide for sufficient replication time for such bacteria as are being proliferated. Control of these variables is well within the abilities of those skilled in the art of microbiology and industrial processes.

Valve 121 allows the mixture from proliferation/concentration tank 107 to then be applied to reactor 101, recycled back through tank 107 in order to increase the phage concentration, or stored in tank 109 for later utilization. The outflow from proliferation/concentration tank 107, the inflow of target bacteria from tank 105 and growth media from tank 108, the amount of recycle in tank 107, and the residence time in tank 107 may all be adjusted to achieve the desired concentration and quantity of virulent phages. Adjustment of these parameters is well within the ability of those of ordinary skill in the arts of microbiology and industrial processes.

For purposes of the discussion following, the phage panel(s) assembled using this method is considered a custom assembled “standard phage panel(s).” The only difference is that the phage(s) need not be identified for assembly of the panel(s).

### Optional Filtration for Complete Isolation of Desired Wild Phages

At times and for certain applications it may be desirable to more or less completely isolate the indigenous phages virulent for the unwanted bacteria, such as if it is desired to store the phages in a concentrated or inactive form, or to isolate the phages for further analysis and study. Therefore, an alternate embodiment of the indigenous phage isolation and proliferation system previously described would include subsystem 192 (delineated by dotted lines and marked 192), which provides for additional filtration to separate indigenous phages virulent for the unwanted bacteria from the other indigenous phages isolated from the biomass.

In this embodiment, the mixture from incubation tank 104 is directed by valve 117 through TFF 134, a microfiltration TFF intended to allow phages to pass through the pores. As discussed previously, the mixture from incubation tank 104 consists of indigenous phages isolated from the biomass in reactor 101 and of unwanted bacteria from tank 105. Some percentage of the indigenous phages virulent for the unwanted bacteria will have infected the unwanted bacteria. Thus, as TFF 134 separates the unwanted bacteria from the unadsorbed phages, those phages attached to the unwanted bacteria will accompany the bacteria into the retentate. All other phages will be removed in the filtrate and removed through conduit 129 to be discarded or used in another application.

The retentate of TFF 134 will pass through container 106 and three-way valve 118 will then direct the retentate back through the TFF. Recirculation will be performed to substantially remove all unadsorbed phages from the mixture, so that only bacteria remain. Selecting values for recirculation duration and other parameters is well within the abilities of those skilled in the art of microbiology.

Once the bacteria and phages are sufficiently separated, valve 118 will then direct the resulting retentate (i.e. the unwanted bacteria, some of which are infected with phages virulent for the unwanted bacteria) through three-way valve 119, which will direct the retentate either into proliferation/concentration tank 107 or storage tank 110. The lytic cycle of the phages will then complete and the infected bacteria burst, yielding phages virulent for the target bacteria. In another embodiment, the resulting mixture will then be further separated to create an isolate of indigenous phages virulent for the unwanted bacteria, which may then be stored or used, as suitable to the application.

In an alternative embodiment, the phages may be isolated from any portion of the biomass stream (not just biomass taken from the bioreactor). Some portion of the biomass may be taken before the biomass enters the reactor, after it leaves the reactor, or any other part of the biofuel production system. So long as the biomass contains phages virulent for at least some of the unwanted bacteria, it may be used. In another embodiment, phages applied to the biomass (for example, from a standard phage panel), and phages that are a result of applied phages (for example, progeny of applied phages), may be recaptured and used as resident phages as described herein.

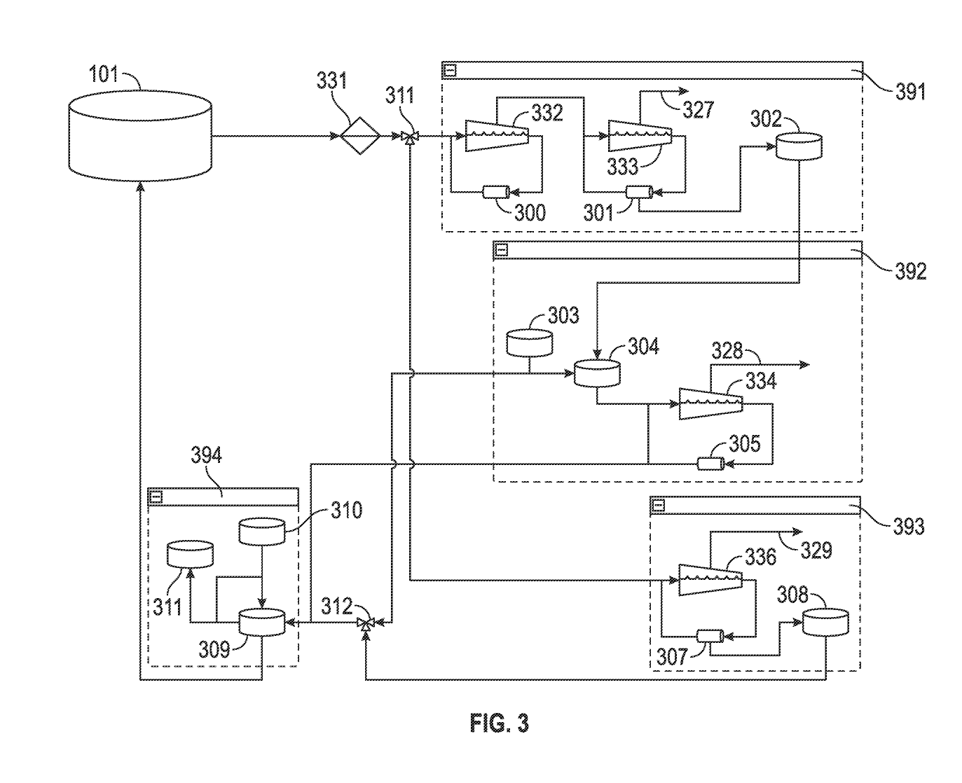

Isolation and proliferation of indigenous phages without identification of unwanted bacteria. Alternatively, when the system organism is known, indigenous phages can be proliferated directly from the target biofuel production process without identifying the unwanted bacteria. One embodiment of this aspect of the invention is illustrated in FIG. 3. Biomass stream is diverted from reactor 101 through filter 331 to remove debris and “trash.” The filtrate, which includes bacteria—unwanted and system (wanted)—and phages indigenous to the reactor, passes through three-way valve 311 into sub-system 391(delineated by dotted lines and marked 391) for isolation of all indigenous phages.

In sub-system 391, the filtrate passes into TFF 332, a micro-filtration TFF intended to isolate the phages from the bacteria. The retentate is recycled through container 300 and back through TFF 332, while the filtrate, containing the wild phages indigenous to the reactor, is passed to TFF 333 for concentration.

The filtrate from TFF 333 is water, and is removed through conduit 327 to be discarded or used in another application. The retentate from TFF 333 is recycled through container 301 and back through TFF 333 for sufficient duration to create a phage solution of the desired concentration, before being passed into holding tank 302. In an alternative embodiment, TFF 333 can be eliminated and the filtrate of TFF 332 passed directly to holding tank 302. The resulting phage solution will be less concentrated than if TFF 333 is used. This embodiment may be desirable in cases when reduction in time, labor, and/or expense is required.

The solution of indigenous phages from holding tank 302 is then passed to sub-system 392 Delineated by dotted lines) for isolation of indigenous phages virulent for the unwanted bacteria. The phages from holding tank 302 are passed into incubation tank 304. Externally cultured system bacteria are added from tank 303. The indigenous phages and the system bacteria are incubated in tank 304 for a time sufficient for adsorption, but not sufficient for completion of the lytic cycle (in particular, not sufficient for lysis of the cells and release of the phage progeny), before being passed to TFF 334, a microfiltration TFF designed to separate phages from bacteria. The filtrate contains phages, which are removed via conduit 328 to be discarded or used in another application. The retentate is recycled through container 305 and back through TFF 334. Recirculation will be performed to substantially remove all unadsorbed phages from the mixture, so that only bacteria remain. Selecting values for recirculation duration and other parameters is well within the abilities of those of ordinary skill in the arts of microbiology and industrial processes. The resulting retentate is a mixture largely comprised of system bacteria, some of which will have virulent phages adsorbed to them, and is passed to sub-system 394 for proliferation.

The filtrate from filter 331 is also diverted by three-way valve 311 into sub-system 393 (delineated by dotted lines) for isolation of indigenous bacteria. The filtrate is passed through TFF 336, a micro-filtration TFF used to separate bacteria and phages. The filtrate, containing phages indigenous to the reactor 101, are removed via conduit 329 to be discarded or used in another application. The retentate, containing bacteria and unfiltered phages, is recycled through container 307 and back through TFF 336. Recirculation will be performed to substantially remove all un-adsorbed phages from the mixture, so that only bacteria remain. Selecting values for recirculation duration and other parameters is well within the abilities of those of ordinary skill in the art of microbiology and industrial processes. The resulting retentate is comprised chiefly of all bacteria indigenous to the reactor 101, and is passed into storage tank 308. The contents of storage tank 308 are passed to sub-system 394 (delineated by dotted lines) to supply hosts for proliferation of phages, virulent for unwanted bacteria, isolated in sub-system 392. In an alternative embodiment, the bacteria from conduit 327 may be used in place of or in conjunction with all or part of sub-system 393.

The mixture of all bacteria indigenous to the reactor, isolated in sub-system 393, and the phages virulent for the unwanted bacteria, isolated in sub-system 392, are both combined in sub-system 394 in proliferation/concentration tank 309. Growth media from storage tank 310 is added as necessary to proliferation/concentration tank 309. The resulting mixture is distributed to one or more of: storage tank 311, reactor 101, and one or more treatment vessels. Additionally, a portion of the outflow may be re-circulated back into proliferation/concentration tank 309 in order to increase the concentration of phages. The volume recycled and the residence time in tank 309 may be adjusted to control concentration and to provide for sufficient residence time for such phages as are being proliferated. Control of these variables is well within the abilities of those of ordinary skill in the arts of microbiology and industrial processes.

Proliferating indigenous phages without identification, and without isolating only those phages virulent for a specific strain(s) of unwanted bacteria, provides a number of benefits. Because the unwanted bacterial strain(s) need not be identified, time, labor, and expense is saved. As resident phages normally exist alongside their host bacteria, if the unwanted bacteria change over time, the proliferated phages will also change, because the system simply proliferates and increases the concentration of the phages already present, so long as they are not virulent for the system bacteria. This is especially useful because of the dynamic nature of microorganism populations—that is, as the concentration of the originally targeted unwanted bacterial strain(s) is reduced, it is possible and even likely that other strain(s) will be free to increase in concentration. In this embodiment, the treatment, therefore, is also dynamic: as the bacterial populations shift, so does the phage panel(s). For this reason, it may be termed a “dynamic phage multi-panel.”

It is necessary to periodically empty the proliferation/concentration and storage vessels, as it is not possible to completely remove all indigenous phages virulent for the system bacteria. Therefore, these phages will reproduce, especially when a mixture of all bacteria (including system bacteria) are used as hosts during proliferation/concentration, although with less effect on bacteria and likely at a slower rate, due to their significantly smaller concentration compared to the phages virulent for the unwanted bacteria. As the process continues, and their concentration increases, the affected mixtures must be discarded and re-isolated in order to keep the concentrations of phages virulent for system bacteria at sufficiently depressed levels.

### Purification of Dynamic Phage Multi-Panel

In an alternative embodiment, the dynamic phage multi-panel is purified and optionally concentrated before proliferation and/or application to the biomass stream. In this embodiment, the multi-panel is passed through a filtration system (such as one or more filters to remove debris and “trash”, followed by a microfiltration tangential flow filter to separate bacteria and phages), to isolate the phages from the remainder of the biomass. Optionally, the multi-panel may also be concentrated, such as by an ultra-filtration tangential flow filter. These steps may be performed before further proliferation or before application, although prior to proliferation is preferred for more efficient utilization of filters. This embodiment may be useful in situations where it is desirable to prevent the inadvertent proliferation of microorganisms or agents that may be harmful to the biofuel process or reaction, or for storage of the dynamic multi-panel.

Dynamic Phage Multi-Panel in Conjunction with a Standard Phage Panel(s)

The dynamic phage multi-panel can be used in place of or in conjunction with the standard phage panel(s) described previously. In a particular embodiment of the invention, a pre-assembled standard phage panel(s) may be used to respond rapidly to an infestation, while the dynamic phage multi-panel is setup and production increased to a suitable output and concentration. In another embodiment, a custom-assembled standard phage panel(s) would be used in conjunction with the dynamic phage multi-panel. This embodiment may be used when it is desired for the custom-assembled standard panel(s) to specifically target the identified unwanted bacteria, ensuring therapy was focused on those species, while the dynamic multi-panel serves to both a) reduce the quantity of custom-assembled panel necessary by virtue of producing some level of phages targeting the same bacteria and b) hold in check other, non-identified or non-targeted species as described previously.

### 4. EXAMPLES

A set of experiments illustrate the ability to identify and reduce the level of unwanted LAB bacteria in fermentation system processing vcorn mash to ethanol.

### Example 1

A Series of LAB strains were isolated from biofuel ethanol fermentation plants. Of a total of 18 samples obtained from two commercial biofuel ethanol fermentation plants two were chosen for study of effects upon acid production that was disruptive to ethanol production—Fermenter samples after 11 hours and after 24 hours from Plant A. Dilution series of either 1 ml of each liquid and solid sample (following elution of bacteria from 1 gm of solid material into 10 ml of PBS) were prepared in sterile phosphate buffered saline solution and spread onto MRS plate. The plates were incubated anaerobically.

To isolate clonally pure LAB, well-separated colonies were picked and subject to several rounds of colony purification. The identity of select purified isolates were determined based on comparing the 16s rRNA coding region to sequences in the public database. This was accomplished by isolating DNA from a colony, PCR amplification of the 16s rRNA coding region, followed by purification and BigDye terminator sequencing the PCR product. All isolated bacteria were determined to be lactic acid bacteria, including Streptococcus equinus, Enterococcus sp., Bifidobacterium thermophilum, Pediococcus pentosaceus, Lactobacillus plantarum, L. mucosae, L. coryniformis, and L. fermentum.

Because comparability few bacterial species actually grow in culture, culture based bacterial identification methods do not in fact provide an accurate portrait of bacterial species from most samples. Therefore, in addition to determining the identification of bacteria cultured from the fermentation plant samples, a culture-independent approach was used to evaluate the population of bacteria at the plant. This approach was based on shotgun pyrosequencing of bacterial 16s rRNA coding region PCR products. Total DNA was isolated from two samples were processed: the 11 hour and 24 hour fermenter samples from Plant A, the 16s coding region amplified by PCR, and subject to bacterial tag-encoded FLX amplicon pyrosequencing (bTEFAP). Resulting sequences were trimmed and quality scored. All sequences passing quality score were compared using BLASTn to a ribosomal database to make taxonomic classifications.

A total of 37,397 bacteria were assayed corresponding to 10,209 and 27,188 individual bacteria from the 11 hr and 24 hr samples, respectively. These bacteria could be organized into sequence clusters, or operational taxonomic units, which correlates to the number of species. A total for 120 different species were identified in the two samples, 70 species in the 11 hr sample and 100 species in the 24 hr sample (Table 5). Only 30 species were found in both samples. 108 species were categorized as “low abundance”, e.g. less than 100 individuals for each sample. The 12 most abundant species were found in both samples and constituted 95% and 97% of all the bacteria identified in the 12 hr and 24 hr samples, respectively. Seven of the thirteen species were Lactobacillus species, including Lactobacillus fermentum, L. johnsonii, L. mucosae, L. reuteri, L. rhamnosus, and L. vaginalis. The other bacteria identified as the most abundant bacteria are not known to be acid producing and as such are not considered to be problematic.

TABLE 1
Culture independent analysis of the thirteen most abundant bacterial
species present in Plant A samples 7 hour and 24 hour.

| | 7 HR, | 24 HR, | 7 HR, | 24 HR, |
|---|---|---|---|---|
| Species | % | % | counts | counts |
| Aquimarina sp | 67.7 | 39.7 | 6916 | 10791 |
| Pseudomonas sp | 23.0 | 16.1 | 2343 | 4384 |
| Lactobacillus mucosae | 0.8 | 18.8 | 86 | 5107 |
| Lactobacillus reuteri | 0.6 | 13.0 | 57 | 3538 |
| Lactobacillus sp | 1.6 | 4.4 | 159 | 1189 |
| Lactobacillus johnsonii | 0.1 | 2.9 | 12 | 799 |
| Caloramator sp | 0.7 | 0.5 | 75 | 135 |
| Lactobacillus vaginalis | 0.1 | 0.6 | 11 | 160 |
| Lactobacillus fermentum | 0.0 | 0.5 | 3 | 148 |
| Prevotella sp | 0.3 | 0.4 | 26 | 118 |
| Alkaliflexus sp | 0.9 | 0.1 | 89 | 31 |
| Lactobacillus rhamnosus | 0.1 | 0.3 | 15 | 91 |
| total | 95.9 | 97.4 | 10,209 | 27,188 |

Shown is the % of population and the actual number of sequence reads corresponding to that species (counts).

Importantly, the identification of Lactobacillus species as the numerically dominant acid producing bacteria in these fermentation samples strongly supports the contention that Lactobacillus is a critical genera to target by phage application.

### Example 2

With the successful collection of phages as described in Example 1 active against the biofuel ethanol fermentation-inhibiting Lactobacillus strains, experiments designed to test the efficacy of phage in controlling LAB were conducted. Batch culture growth follows classic, single-step growth kinetics and can be broken down into three phases, lag, log, and stationary phase. Lag phase corresponds to the early, acclimation of the inoculum to fresh growth media, log (logarithmic or exponential) phase corresponds to the period of most rapid bacterial cell division, and stationary phase corresponds to when limiting nutrients are depleted and cell division rates decline or cease. Bacterial growth was monitored by changes in optical density (OD₆₀₀ₙₘ) with OD₆₀₀ₙₘ=1 of Lactobacillus sp. corresponding to approximately 1×10⁸ cfu/ml (cfu=colony forming units, a measurement of the number of viable cells).

The LAB control experiment was conducted using host L. fermentum strain 0315-25 and four phages (25Soila, 25Sau, 25Wang, and 25Komiso). Nine parallel cultures were set up. All nine cultures received the same bacterial inoculum and incubated at 37° C. until early-mid log growth phase. At this point, phage was added either singly or combined into phage cocktails. The multiplicity of infection (moi, or the ratio of phage to bacterial cells) was adjusted to 0.1, 1, 5 or 10. Bacterial growth was monitored for 27 hours to measure the effect of the phage treatment. The nine cultures were incubated until the culture OD₆₀₀ had increased from the initial inoculum of less than 0.1 to around 0.5, at which time 8 of the nine cultures received phage lysate and the ninth, control culture was treated with an equal volume of MRS broth. Due to dilution, there was an immediate drop in the OD₆₀₀ of all nine cultures to about 0.3. However, within minutes after the addition of phage, the control culture was showing signs of recovery while the phage treated cultures did not. By 15 minutes after addition of phage at moi of 5 or 10, the culture OD₆₀₀ dropped to below the detection limit. The cultures treated with the lower phage moi of 0.1 and 1 also exhibited a drop in OD₆₀₀ to below the detection limit, although the process took between 5 and 10 hours. At the end of the testing period, the control culture reached stationary stage at an OD₆₀₀ between 1.0 and 1.2, while all of the phage treated cultures remained “clear”, with OD₆₀₀≦0.000, at 27 hours following treatment.

The batch culture phage efficacy test clearly demonstrated that phage treatment controlled bacterial growth for at least 27 hours. Because phage were demonstrated to control bacterial growth in pure culture, experiments testing the capacity of this phage preparation to control bacterial growth in a mixed culture with the fermentative yeast were conducted.

### Example 3

Another experiment was conducted to determine phage efficacy on controlling LAB in a shake-flask fermentation contamination model system (Bischoff et al., 2009). For each experiment, five shake-flask cultures were set up with a corn mash feedstock inoculated with Saccharomyces cerevisiae to an initial OD₆₀₀=1. Four of the S. cerevisiae cultures were contaminated with L. fermentum 0315-25 at a density of 10⁷ cfu/ml (yeast+LAB). Three of the LAB contaminated yeast cultures were treated with phages 25Sau and 25Inf, either singly or in combination, at moi of 1, 20, and 0.5 and 10, respectively. The entire experiment was set up in triplicate and the data are reported as the mean value±SEM for triplicate cultures (Table 2).

TABLE 2
LAB phage efficacy trial in a fermentation contamination model system.

| | Ethanol | Glucose | Lactic Acid | Acetic Acid |
|---|---|---|---|---|
| A. Yeast | 137 +/− 2.4 | 0.44 +/− 0.19 | 1.9 +/− 0.05 | 0.85 +/− 0.04 |
| B. Yeast + LAB | 117 +/− 1.6 | 28.7 +/− 5.7 | 5.3 +/− 0.13 | 2.8 +/− 0.07 |
| C. Yeast + Lab + Phage Sau | 137 +/− 1.0 | 0.47 +/− 0.13 | 2.4 +/− 0.01 | 0.76 +/− 0.03 |
| D. Yeast + Lab + Phage Inf | 134 +/− 0.9 | 0.42 +/− 0.13 | 2.4 +/− 0.07 | 0.84 +/− 0.03 |
| E. Yeast + Lab + Phage Sau + Inf | 136 +/− 0.9 | 0.60 +/− 0.09 | 2.4 +/− 0.04 | 0.76 +/− 0.03 |

For Table 2, the amount of ethanol, glucose, lactic acid, and acetic acid were determined at the end of a 72 hour fermentation in corn mash with S. cerevisiae only (Fermentation control, A.), S. cerevisiae and LAB co-culture (Contamination control, B.), or the S. cerevisiae and LAB co-culture treated with phage 25Sau (C.), 25Inf (purple), individually or 25Sau+25Inf together (D.). Values are in g/L and are the average of triplicate experiments, with the indicated standard deviations.

The effect of phage treatment compared to no treatment was determined by measuring levels of ethanol, glucose, lactic acid, and acetic acid were determined after 72 hours of incubation. These measurements were made using high performance liquid chromatography (HPLC) using a 300 mm Aminex HPX 87H column (Bio-Rad Laboratories, Inc., Hercules, Calif.) using 10 ml of sample injected onto a heated column (65° C.) and eluted at 0.6 ml/min using 5 mM H₂SO₄ as mobile phase. Concentrations were reported as mean values (±standard deviation) of triplicate cultures. Statistical comparisons of challenged and control cultures were performed using Student's t-test (P<0.05) (FIG. 5). The concentration of phage was analyzed at three times during the experiment: at 7, 18, and 24 hours and found to vary between 10⁵ and 10⁸ pfu/ml. The fermentation model experiment was also conducted using a lower initial inoculum of LAB and phage at 10⁶ cfu/ml. The lower LAB inoculum level did not inhibit ethanol production to the extent that the 10⁷ inoculum level did, however the phage treatments recovered ethanol yields to the same level as the uncontaminated yeast culture.

The fermentation model clearly demonstrated the capacity that phage have to prevent ethanol yield loss in the presence of Lactobacillus.

### 5. APPLICATION OF PHAGE PANEL(S)

Application of the phage panel(s) for control of the unwanted bacteria is accomplished in a variety of ways, four of which are described below. These are: 1) pre-loading of the biomass stream before reaction, 2) continuous treatment of the reacting biomass for prevention purposes or for treatment of chronic bacterial infestations, 3) acute treatment of the reacting biomass for sudden bacterial overgrowth, and 4) treatment of an empty reactor before reaction begins. These four methods may be combined in any combination, as necessary for the particular plant and application and use of the invention.

### 1) Pre-Loading

In this aspect of the invention, incoming biomass stream is pre-treated before it enters a reactor. In one embodiment, shown in FIG. 1, valve 126 diverts some or all of the incoming biomass stream from previous production step 181 through phage treatment vessel 109 before it enters reactor 101. After incubation for a suitable time, as described below, the treated biomass flows through valve 123 into the reactor 101. This pretreatment of the biomass stream before it enters the reactor “pre-loads” the biomass stream with phages, allowing the phages to begin adsorption and infection of the bacteria before the reaction process begins.

If insufficient bacteria are present for the infection rate to be significant, the phages will remain in the now-reacting biomass. Because phages are relatively hardy, especially in an environment in which their host bacteria thrive, they will persist in the reacting biomass as long as the biomass remains in the reactor. If concentrations of unwanted bacteria are low, the infection rate will also be extremely low, but the few infections that do occur will only increase the number of phages present. With a large quantity of phages residing in the reacting biomass, rising concentrations of bacteria will result in rising infection rates and, thus, rising phage concentrations, thereby preventing an acute infection—before production efficiency is negatively impacted.

### 2) Continuous Treatment

In another aspect of the invention, the biomass residing in a reactor is treated continuously. In one embodiment, shown in FIG. 1, some portion of the volume of reactor 101 flows through valve 113 into phage treatment vessel 109. After incubation for a suitable time, as described below, the treated biomass flows through valve 123 into the reactor. Alternatively, concentrated phage mixture may be delivered directly to the reactor from phage treatment vessel 109 (through valve 123) and/or phage storage tank 110 (through valve 124).

Continuous treatment of the reacting biomass can be used to 1) address chronic infections of the reaction process, and 2) prevent infection, such as in plants that regularly deal with sudden, acute overgrowths. The rationale for “pre-treating” the biomass is detailed in the preceding section. Continuous treatment is a natural extension of “pre-loading” the biomass stream, such as in situations in which pre-loading was not entirely sufficient in itself to provide a suitable concentration of phages, or in which phage concentration is decreasing due to increasing volume.

### 3) Acute Treatment

In another aspect of the invention, the reacting biomass is treated with a sufficient quantity of phages to rapidly increase phage concentration. In one embodiment, shown in FIG. 1, a large dose of concentrated phage solution is delivered to reactor 101 from storage tank 110 (through valve 124) and/or from the phage proliferation/concentration tank 107 (through valve 121) and/or from phage treatment tank 109 (through valve 123). To provide the quantity necessary, the phage treatment may be supplemented by adding phages from an external location, possibly delivered in a concentrated liquid or dried form, as discussed previously.

Alternatively, the process described above for continuous treatment may be modified to treat all, or at least a greater portion, of the reacting biomass by increasing the percentage of the reacting biomass that is circulated through the phage treatment tank, and/or by adding multiple treatment tanks. Multiple concentration and/or treatment tanks may also be used in other aspects of application, concentration, or proliferation in order to increase biomass treatment and phage production capacity.

Delivering a large quantity of phages to the reacting biomass can be used to treat the acute bacterial overgrowths responsible for substantially retarded reactions, such as “stuck” fermentations. Rapid increase of phage concentrations in biomass with a large concentration of unwanted bacteria will cause rapid infection and lysis of the unwanted bacteria. As the lytic cycle completes, the phage concentrations will increase as the bacterial concentrations decrease, until the bacteria population is reduced to acceptable levels and the reaction process can proceed without interference.

### 4) Equipment Treatment

In another aspect of the invention, concentrated phage mixture in a liquid form is used to treat plant equipment. One embodiment of this aspect is depicted in FIG. 1, where phage panel(s) from tank 110 (through valve 124), and/or phage mixture from phage proliferation/concentration tank 107 (through valve 121) and/or phage treatment tank 109 (through valve 123), is delivered into reactor 101. The mixture is left to incubate for a period sufficient for infection and lysis of unwanted bacteria, and flushed to waste or returned to a storage tank or treatment tank. Methods to reduce the quantity of phages needed for treating equipment may include spraying the phage mixture onto the inner tank walls rather than completely filling the tank, and/or incorporating the phages in a medium that will cause them to remain on the tank and equipment.

Phage treatment of plant equipment, particularly the reactor, pipelines, and related equipment, will reduce unwanted bacteria levels residing on or in the equipment. The equipment may be treated between every reaction batch, or on a schedule based on time, number of batches processed, or volume of biomass processed. Regular treatment will prevent the buildup of unwanted bacteria and the infestation of new batches with unwanted bacteria from previous batches, especially if coupled with a regular regimen of mechanical cleaning. Alternatively, if a continuous production scheme is used (as opposed to batch production), phage treatment may be incorporated into a regular preventative cleaning schedule, or as part of a cleaning/disinfection process after serious bacterial infestations.

### Incubation of Phage Solution

In the phage proliferation/concentration tank and in any treatment applications, the phage panel(s)/mixture must have a residence time sufficient for the phages to progress to the desired stage of the lytic cycle. Treatment is not complete until the lytic cycle is complete—starting with adsorption (initial attachment) of the phages to the bacteria, progressing through infection of the bacteria and replication of the phages, and ending with lysis of the bacterial cells. In some cases, the phage mixture must reside at the point of application until the entire cycle is completed. This is the case for treatment of an empty tank, as described above.

In some cases, however, residence time of the biomass can be only slightly longer than that sufficient for adsorption and firm attachment of the phages to the bacteria. This would be the case, for example, in the incubation tank (104 in FIG. 1) when using subsystem 192, where completion of the lytic cycle would prevent separation of the desired phages (those virulent for the unwanted bacteria) from the undesired phages (especially those virulent to the system bacteria). In other cases, residence time of the biomass at the point of application also need only be sufficient for adsorption and firm attachment of the phages to the bacteria, although longer residence times may not necessarily be detrimental. This would be the case, for example, in the treatment tank (109 in FIG. 1) or in the proliferation/concentration tank (107 in FIG. 1).

Once the phages have attached to the bacteria, the lytic cycle will progress to completion regardless of the location. In the last example immediately preceding, as long as a sufficient portion of the contents of the phage concentration tank are recycled so that the tank is replenished with phages and, if desired, the concentration is rising, there is no need for the mixture to reside in the tank for the entire lytic cycle. This process will reduce residence time, thereby increasing throughput and process efficiency.

During treatment (i.e. some or all of the biomass stream passes through a phage treatment tank, whether before or after entering the reactor, and then passes into the reactor), releasing the treated biomass back into the reactor before the lytic cycle completes will cause the infected bacteria to burst inside the treatment tank. This will result in a type of continuous treatment, as described in the relevant section above.

### Treatment Tank

As can be inferred from the descriptions, a separate treatment tank(s) is required in the embodiments described herein. If biomass were passed directly through the proliferation/concentration tank, wild phages in the biomass that are virulent for the system bacteria would be inadvertently proliferated, and could rapidly reach levels detrimental to the process in the bioreactor. Thus, a separate treatment tank(s) is required to prevent the unfiltered biomass from the bioreactor from contaminating the phage panel(s) in the proliferation/concentration tank. Alternatively, as discussed previously, the treatment tank may be the reaction vessel itself.

The treatment tank would be supplied with concentrated phage mixture from either the phage concentration tank, the phage panel(s) storage tank, or from an outside source of phages, and biomass would be passed through the treatment tank. An additional advantage of a separate treatment tank is that it allows the phage proliferator/concentrator to be operated at a rate independent of the treatment flow rate required.

### Suitable Concentration Levels of Bacteria and Phages

The simple fact that phages virulent for a certain bacteria are present at any given concentration (where phage concentrations are measured in “plaque forming units,” or PFU) does not mean that they will necessarily be effective at reducing bacterial concentrations (where bacterial concentrations are measured in “colony forming units,” or CFU) at that phage concentration. Like most predators, phages are designed to proliferate alongside their host, without completely destroying their host population. Therefore, unlike most antibiotics and other chemicals, increasing phage concentration levels does not necessarily correspond to increased efficacy or even increased destruction of bacteria.

In general, however, a MOI (ratio of infectious agents—phages—to infection targets—targeted bacteria), on the order of one (1) is considered effective, with at least ten being preferred. In the case of LAB in corn ethanol yeast-based fermentation, concentrations of bacteria from 10⁵ to 10⁹ CFU/mL have been shown to have significant negative impact on ethanol production (Bischoff, Liu et al. 2009), and concentrations of phages between 10⁴ and 10⁹ PFU/mL are considered feasible to produce when dealing with LAB virulent phages, with 10⁶ to 10⁹ PFU/mL being preferred for increased reaction rates.

Therefore, in one embodiment of this invention, the phage panel(s) application methods described previously apply phages such that the target MOI at the point of treatment is at least one (1), with at least ten (10) being preferred. In a preferred embodiment, the invention is focused on controlling LAB in bacterial-based corn ethanol fermentation, and the phage panel(s) application methods described previously apply the panel(s) at concentrations of at least 10⁴ PFU/mL, with at least 10⁵ PFU/mL being preferred, and 10⁶ PFU/mL being especially preferred.

### Concentration of Unwanted Bacteria in Reacting Biomass

It may be necessary at times to increase the rate of phage adsorption and infection of the unwanted bacteria. This may occur when the concentration of unwanted bacteria is high enough to reduce efficiency, but low enough that adsorption and infection is occurring at an insufficient rate, as discussed above. This can be accomplished by increasing the concentration of phages at the point of application. If phage production is dependent on the concentration of bacteria present in the biomass, however, it may not be possible to produce a sufficient quantity of phages at a rate sufficient for treatment. Therefore, it is desirable in some situations to increase the concentration of bacteria in the biomass in order to increase phage proliferation.

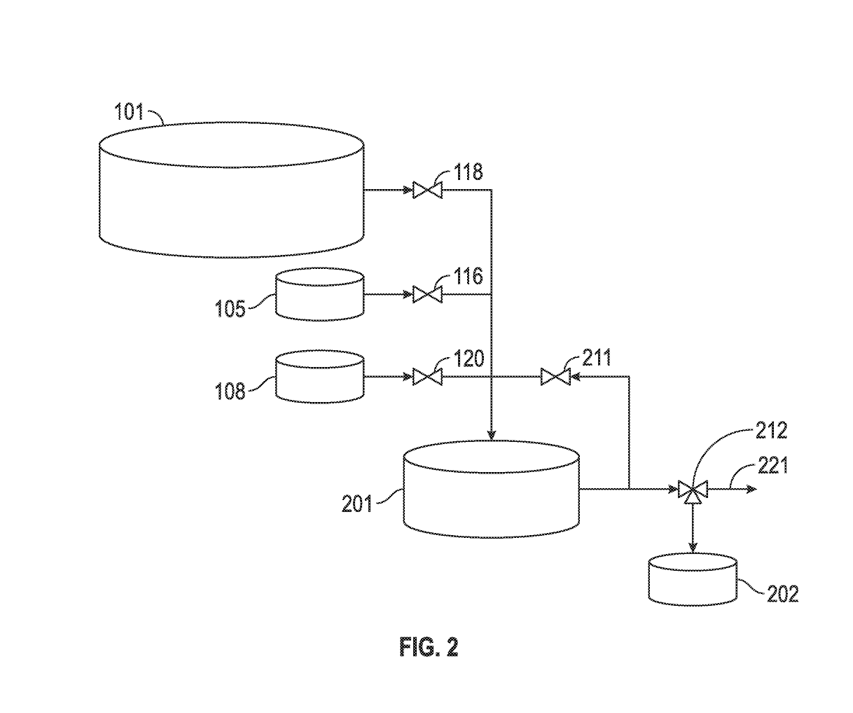

In another aspect of the invention, therefore, a continuous bacteria concentration/proliferation system is included in the system described above. An embodiment of this aspect is in FIG. 2. Reacting (for example, fermenting) biomass from reactor 101 is fed into bacteria proliferation/concentration tank 201 through valve 118. Initial bacteria may be supplied from storage tank 105 through valve 116. Growth media suitable for the unwanted bacteria, such as MRS or Rogosa SL media for Lactobacilli species, is added to the bacteria proliferation/concentration tank from tank 108 through valve 120. Valve 212 directs the flow from the bacteria proliferation/concentration tank to a storage tank 202 and/or through conduit 221 to the phage proliferation/treatment system. A portion of the outflow is recycled through valve 211 back into the bacteria proliferation/concentration tank to continue the bacteria proliferation process and to increase the concentration of bacteria in the tank. Residence time in tank 201 is adjusted for the specific strain(s) of unwanted bacteria to maximize proliferation. This aspect is also embodied in FIG. 1, wherein some portion of the contents of tank 105, containing unwanted bacteria, is recycled through valve 125 back into tank 105. Growth media from tank 108 is directed by valve 120 into tank 105 as necessary.

Alternatively, the concentration of the unwanted bacteria in the treatment tank may be rapidly increased by adding unwanted bacteria directly from a storage or proliferation/concentration vessel, such as tank 105, to the application point, such as treatment tank 109. Although this appears to be counterintuitive, the goal is to raise the concentration of unwanted bacteria at the point of application to a level sufficient for the infection rate of the unwanted bacteria by the phages to increase to a level sufficient for treatment. As the treatment continues, the concentration of phages will increase as more host bacteria are infected, used to reproduce phages, and lysed. Conversely, the concentration of bacteria will decrease to levels below that at the beginning of treatment, and below that at which the unwanted bacteria are interfering with the reaction process.

### Monitoring Unwanted Bacteria

It is desirable to monitor the levels of unwanted bacteria before, during, and after application of phage panel(s) in order to maximize treatment efficacy and efficiency. Therefore, an embodiment of the invention incorporates one or more means to monitor the presence and/or concentration (absolute or relative) of target unwanted bacteria. Suitable means include, but are not limited to: taking biomass samples at regular intervals, and using standard microbiological techniques in an on- or off-site laboratory; utilizing “rapid field assays,” such as those offered by ETS Labs; or monitoring of levels of known metabolites, such as lactic or acetic acid.

In a preferred embodiment, a bacterial-based fuel ethanol fermentation system uses a combination monitoring approach comprised of: constant or frequent monitoring of levels of metabolites of unwanted bacteria (such as lactic acid for LAB or acetic acid for AAB), regular usage of rapid field assays, with occasional laboratory analysis of samples. This multi-faceted approach provides a combination of the feasibility and shorter lag-times of rapid monitoring techniques (metabolites and field assays) with the greater accuracy and precision of laboratory analysis. Thus, this embodiment provides both rapid monitoring of daily conditions and fluctuations, and monitoring for additional longer-term problems and warnings, and the treatment program may be adjusted accordingly.

Application in Conjunction with Other Control Methods

In an embodiment of this invention, phages are applied in conjunction with one or more other means of controlling unwanted bacteria, such as antibiotic, biocide, or antimicrobial therapies. In this embodiment, phage treatment can be applied at alternating times with the selected additional treatment(s), in phases (for example, initial treatment with phages and follow up treatment with antibiotics, or vice versa), or simultaneously. Such treatment options may be desirable, for example, during phage production when the phages produced are not yet sufficient for sole therapy; or when antibiotic therapy is still desired, but phage therapy is employed on a rotational basis to reduce antibiotic usage and development of resistance to antibiotics. Selection of suitable therapies and combinations thereof is well within the abilities of one of ordinary skill in the art of microbiology and biological industrial process management.

### Another Preferred Embodiment

In a preferred embodiment of the invention described, the bioreaction is a fermentation process for the production of fuel, the bioreactor is a tank (and any related apparatuses or assemblies) used in industrial fermentation, and the biomass is a feedstock, or any downstream, processed portion of the original feedstock. In an especially preferred embodiment, the invention is applied specifically to ethanol production. The feedstock in this embodiment may be any feedstock suitable for fermentation (before, during, or after any additional treatment steps) in the process of producing ethanol. Suitable feedstocks include, but are not limited to both sugar/starch and cellulosic/lignocellulosic feedstocks: grains (such as corn, wheat, milo, barley, millet), sugar cane, sugar beets, molasses, whey, potatoes, agricultural residue (crop residues such as bagasse, wheat straw and corn stalks, leaves, and husks), forestry residue (logging and mill residues such as wood chips, sawdust, and pulping liquor), grasses (hardy, fast-growing grasses such as switchgrass grown specifically for ethanol production), municipal and other wastes (plant-derived wastes such as household garbage, paper products, paper pulp, and food-processing waste), and trees (fast-growing trees such as poplar and willow grown specifically for ethanol production) (http://www.afdc.energy.gov/afdc/ethanol/feedstocks.html).

### Deactivation of Phages

Once the biomass stream has passed beyond the process requiring phage treatment, the phages are no longer needed. In most applications, the biomass stream may simply continue down the process flow without further treatment. For instance, in fuel ethanol production, the distillation process will denature phages resident in the biomass, while many of the phages will settle out in the waste sludge. In most cases, the concentration of the phages in the waste streams will not be significantly higher than that of biomass streams which have not undergone phage treatments. Additionally, since phages are highly host-specific, they do not pose a significant health risk to wildlife or human populations coming in contact with them, as is recognized by the GRAS classification of phages by the FDA.

In some cases, however, it may be desirable to reduce the concentration of the phage panel(s) in the biomass stream after treatment. Depending on the phage(s) employed, this may be performed, for example, by a strong denaturing agent (such as sodium hypochlorite), a strong acid, a strong base, or even heat. Therefore, an embodiment of this invention incorporates one or more means of denaturing the phage panel(s) employed. Suitable means may include, but are not limited to: application of a sufficient concentration of denaturing agent, acid, or base to all or some portion of the biomass stream or heating of all or some portion of the biomass stream to a sufficient temperature to denature the phages utilized. The specific parameters will vary largely depending on the phage strain(s) comprising the phage panel(s) employed, and are well within the skill of one skilled in the art of microbiology.

### SCOPE OF THE INVENTION

In this specification, the invention has been described with reference to specific embodiments. It will, however, be evident that various modifications and changes can be made thereto without departing from the broader spirit and scope of the invention as set forth in the appended claims. The specification is, accordingly, to be regarded in an illustrative rather than a restrictive sense. Therefore, the scope of the invention should be limited only by the appended claims.

### REFERENCES

Ackermann, H. W. (2007). “5500 Phages examined in the electron microscope.” Arch Virol 152(2): 227-43.
Bischoff, K. M., S. Liu, et al. (2009). “Modeling bacterial contamination of fuel ethanol fermentation.” Biotechnol Bioeng 103(1): 117-22.
Bischoff, K. M., K. A. Skinner-Nemec, et al. (2007). “Antimicrobial susceptibility of Lactobacillus species isolated from commercial ethanol plants.” J Ind Microbiol Biotechnol 34(11): 739-44.
Bren, L. (2007). “Bacteria-Eating Virus Approved as Food Additive.” FDA Consumer magazine 41(1).
Brownell, G. H., J. N. Adams, et al. (1967). “Growth and characterization of nocardiophages for Nocardia canicruria and Nocardia erythropolis mating types.” J Gen Microbiol 47(2): 247-56.
Brownell, G. H. and J. E. Clark (1974). “Host range alteration of nocardiophage phi C following lysogenization of Nocardia erythrypolis.” J Gen Virol 23(3): 247-54.
Brussow, H. (2001). “Phages of dairy bacteria.” Annu Rev Microbiol 55: 283-303.
Casjens, S. R. (2008). “Diversity among the tailed-bacteriophages that infect the Enterobacteriaceae.” Res Microbiol 159(5): 340-8.
De Alwis, H. and D. N. Heller “Multiclass, multiresidue method for the detection of antibiotic residues in distillers grains by liquid chromatography and ion trap tandem mass spectrometry.” J Chromatogr A 1217(18): 3076-84.
Hatfull, G. F. (2008). “Bacteriophage genomics.” Curr Opin Microbiol 11(5): 447-53.
Housby, J. N. and N. H. Mann (2009). “Phage therapy.” Drug Discov Today 14(11-12): 536-40.
Jones, D. T., M. Shirley, et al. (2000). “Bacteriophage infections in the industrial acetone butanol (AB) fermentation process.” J Mol Microbiol Biotechnol 2(1): 21-6.
Kropinski, A. M. (2006). “Phage Therapy—Everything Old is New Again.” Can J Infect Dis Med Microbiol 17(5): 297-306.
Kurtboke, D. I. and J. R. French (2007). “Use of phage battery to investigate the actinofloral layers of termite gut microflora.” J Appl Microbiol 103(3): 722-34.
Makanjuola, D., A. Tymon, et al. (1992). “Some effects of lactic acid bacteria on laboratory-scale yeast fermentation.” Enzyme Microbial Technol (14): 350-357.
Mattey, M. and J. Spencer (2008). “Bacteriophage therapy—cooked goose or phoenix rising?”Curr Opin Biotechnol 19(6): 608-12.
Mc Grath, S., G. F. Fitzgerald, et al. (2007). “Bacteriophages in dairy products: pros and cons.”Biotechnol J 2(4): 450-5.
Narendranath, N. (2003). Bacterial contamination and control in ethanol production. The Alcohol Textbook. K. A. Jaques, T. P. Lyons and D. R. Kelsall. Nottingham, UK, Nottingham University Press: 287-298.
Narendranath, N. V., S. H. Hynes, et al. (1997). “Effects of lactobacilli on yeast-catalyzed ethanol fermentations.” Appl Environ Microbiol 63(11): 4158-63.
Sabour, P. M. and M. W. Griffiths (2010). Bacteriophages in the Control of Food-and Waterborne Pathogens, ASM Press.
Schell, D. J., N. Dowe, et al. (2007). “Contaminant occurrence, identification and control in a pilot-scale corn fiber to ethanol conversion process.” Bioresour Technol 98(15): 2942-8.
Skinner, K. A. and T. D. Leathers (2004). “Bacterial contaminants of fuel ethanol production.” J Ind Microbiol Biotechnol 31(9): 401-8.
Srinivasiah, S., J. Bhaysar, et al. (2008). “Phages across the biosphere: contrasts of viruses in soil and aquatic environments.” Res Microbiol 159(5): 349-57.
Sturino, J. M. and T. R. Klaenhammer (2006). “Engineered bacteriophage-defence systems in bioprocessing.” Nat Rev Microbiol 4(5): 395-404.
Tao, L., S. I. Pavlova, et al. (1997). “Analysis of lactobacillus products for phages and bacteriocins that inhibit vaginal lactobacilli.” Infect Dis Obstet Gynecol 5(3): 244-51.

1. A process, for control of unwanted bacteria in a biofuel production process comprised of a reaction mediated by bacterial system reactive agents, comprising applying to a biomass substrate an effective amount of bacteriophages virulent for one or more strains of the unwanted bacteria.

2. The process of claim 1, wherein one or more of the unwanted bacteria are selected from the group consisting of species from genera generally known as lactic acid bacteria, including Lactobacillus, Pediococcus, Lactococcus, Enterococcus, Weissella, Leuconostoc, Streptococcus, and Oenococcus, and species from genera generally known as acetic acid bacteria, including Acetobacter and Gluconobacter.

3. The process of claim 1, wherein the biomass is comprised of one or more sugar, starch, cellulosic, or lignocellulosic feedstocks selected from the group consisting of grains, including corn, wheat, milo, barley, millet; sugar cane; sugar beets; molasses; whey; potatoes; agricultural residue, including crop residues such as bagasse, wheat straw and corn stalks, leaves, and husks; forestry residue, including logging and mill residues such as wood chips, sawdust, and pulping liquor; grasses, including hardy, fast-growing grasses such as switchgrass grown specifically for ethanol production; municipal, plant, animal and other wastes, including plant-derived wastes such as household garbage, paper products, paper pulp, and food-processing waste; and trees, including fast-growing trees such as poplar and willow grown specifically for ethanol production.

4. The process of claim 1, wherein at least one of the system reactive agents is selected from the group consisting of: Clostridia, including C. ljungdahlii, C. acetobutylicum and C. thremocullum; Zymomonas mobilis, including any microorganisms modified to include portions of the genetic code of Z. mobilis; Escherichia coli; Bacillus, including B. subtilus and B. coagulans; Acidothermus cellulolyticus; Pseudomonas cellulose; Rhodothermus marinus; and cyanobacteria.

5. The process of claim 1, wherein some portion of the biofuel production process is a member of the group consisting of fermentation of biomass by bacteria; cyanobacteria biofuel production; and production of enzymes from bacteria for immediate or later use in biofuel production.

6. The process of claim 1, wherein the bacteriophages comprise one or more bacteriophage panels such that one or more bacteriophage strains in each panel is virulent for one or more of the unwanted bacteria.

7. The process of claim 6, wherein one or more of the resident bacteriophage strains are included in the panel by continuous proliferation and concentration of bacteriophages, where proliferation and concentration of bacteriophages is facilitated by supplying unwanted bacteria as hosts.

8. The process of claim 6, wherein one or more of the bacteriophage panels are assembled as a standard treatment for one or more application profiles selected from a group comprised of feedstocks, environments, processes, fuel products, geographic locales, system reactive agents, and unwanted bacteria.

9. The process of claim 6, wherein the host bacteria are supplied to the bacteriophages in a sidestream vessel and bacteriophages are delivered to the biofuel production process by delivering some portion of the contents of the sidestream vessel to said biofuel production process.

10. The process of claim 1, wherein bacteriophages are delivered to the biomass directly in a vessel or conduit or in one or more sidestream treatment vessels or conduits, and delivery of the bacteriophages occurs either before or during the biofuel production process targeted for treatment.

11. The process of claim 1, wherein the multiplicity of infection is at least ten (10) and the concentrations of said bacteriophage strains are at least 10⁴ PFU/mL.).

12. The process of claim 1, wherein the bacteria are concentrated in a continuous enrichment procedure in one or more sidestreams.

13. The process of claim 12, wherein a nutrient source, used to feed the unwanted bacteria in the enrichment procedure, is some portion of the biomass.

14. A composition comprising one or more bacteriophages virulent for unwanted bacteria in a biofuel production process in which biofuel production process which is impeded by unwanted bacteria utilizes bacterial system reactive agents.

15. The composition of claim 14, wherein one or more of the unwanted bacteria are selected from the group consisting of species from genera generally known as lactic acid bacteria, including Lactobacillus, Pediococcus, Lactococcus, Enterococcus, Weissella, Leuconostoc, Streptococcus, and Oenococcus, and species from genera generally known as acetic acid bacteria, including Acetobacter and Gluconobacter.
